(19) United States
(12) Patent Application Publication
(10) Pub. No.: US 2023/0149619 A1
(43) Pub. Date: May 18, 2023

(54) HANDHELD PORTABLE ORAL ASPIRATOR

(71) Applicant: Tychermont Products LLC, Philadelphia, PA (US)

(72) Inventor: Tyiwanna Hope Wilson, Philadelphia, PA (US)

(21) Appl. No.: 17/989,755

(22) Filed: Nov. 18, 2022

Related U.S. Application Data
(60) Provisional application No. 63/423,566, filed on Nov. 8, 2022.
(60) Provisional application No. 63/389,072, filed on Jul. 14, 2022.
(60) Provisional application No. 63/280,617, filed on Nov. 18, 2021.

(51) Int. Cl. A61M 1/00

(52) U.S. Cl. CPC A61M 1/65; A61M 1/87; A61M 2210/0625; A61M 2205/8206; A61M 2205/8243; A61M 2205/581; A61M 2205/583

(57) ABSTRACT

A handheld portable oral aspirator device comprising a main unit having a lower portion and an upper portion. The lower portion may include a battery, circuity mounted on a substrate, and a vacuum pump. The upper portion may be configured to house a reservoir coupled to one of a vacuum pump and a vacuum passageway connected to the vacuum pump via a hose. A reservoir cap may be configured to be affixed to one of the reservoir and the upper portion. A flexible mouthpiece may be coupled to the reservoir cap. A handle may be coupled to one or more of the lower portion and the upper portion. The handle may include a trigger that is configured to cause the circuitry to activate the vacuum pump only when pressed.

### CROSS REFERENCE TO RELATED APPLICATIONS

This application claims benefit to U.S. Provisional Application No. 63/280,617 entitled “Handheld Portable Oral Vacuum Liquid Aspirator” filed Nov. 18, 2021, U.S. Provisional Application No. 63/389,072 entitled “Handheld Portable Oral Aspirator” filed Jul. 14, 2022, and U.S. Provisional Application No. 63/423,566 entitled “Handheld Portable Oral Aspirator” filed Nov. 8, 2022 and is related to PCT Application entitled “Handheld Portable Oral Aspirator” filed concurrently on Nov. 18, 2022. The full disclosures of these applications are incorporated herein by reference.

### TECHNICAL FIELD

The present disclosure relates to a device to aid in suction for people with swallowing disorders and/or issues with hypersalivation, and more particularly, a portable handheld oral aspirator to evacuate liquid waste from a person's oral cavity, preferably without outside assistance.

### BACKGROUND

Dysphagia and other oral conditions (e.g., cancer, cleft lip, cleft palate) may cause difficulty in swallowing. Although sometimes classified under “symptoms and signs” of other medical condition, dysphagia may be classified as a condition in its own right. People with dysphagia have difficulty swallowing and may even experience pain while swallowing (“odynophagia”). Some people may be completely unable to swallow or may have trouble safely swallowing liquids, foods, or saliva. When that happens, eating becomes a challenge. Dysphagia can present immediate problems, such as choking and aspiration of fluid into the lungs, and more long-term problems as it makes it difficult to take in enough calories and fluids to nourish the body and can lead to additional serious medical problems. Suction devices are typically used to assist in the removal of saliva and other liquids from the oral cavities of people affected with dysphagia or hypersalivation.

### SUMMARY

A handheld portable oral aspirator device comprising a main unit having a lower portion and an upper portion. The lower portion may include a battery, circuity mounted on a substrate, and a vacuum pump. The upper portion may be configured to house a reservoir coupled to one of a vacuum pump and a vacuum passageway connected to the vacuum pump via a hose. A reservoir cap may be configured to be affixed to one of the reservoir and the upper portion. A flexible mouthpiece may be coupled to the reservoir cap. A handle may be coupled to one or more of the lower portion and the upper portion. The handle may include a trigger that is configured to cause the circuitry to activate the vacuum pump only when pressed. The device may include a charging base configured to receive the lower portion. The charging base may include charging circuitry configured to charge the battery.

### BRIEF DESCRIPTION OF DRAWINGS

Other objects and advantages of the present disclosure will become apparent to those skilled in the art upon reading the following detailed description of exemplary embodiments and appended claims, in conjunction with the accompanying drawings, in which like reference numerals have been used to designate like elements, and in which:

FIG. 1 is a side view of an example handheld portable oral liquid vacuum aspirator device, according to an embodiment of the present disclosure;

FIG. 2 is a side view of another example handheld portable oral liquid vacuum aspirator device, according to an embodiment of the present disclosure;

FIG. 3 is an exploded view of the device, according to an embodiment of the present disclosure;

FIG. 4 is a cross section view of the device, according to an embodiment of the present disclosure;

FIG. 5 is a side view of a reservoir, according to an embodiment of the present disclosure;

FIG. 6 is a side view of another example of the reservoir, according to an embodiment of the present disclosure;

FIG. 7 is a bottom view of a reservoir cap of FIG. 6, according to an embodiment of the present disclosure;

FIG. 8 is a perspective view of the reservoir of FIG. 6 within an upper portion of the device, according to an embodiment of the present disclosure; and

FIGS. 9A-9D are cross section views of different examples of triggers that may be used, according to an embodiment of the present disclosure.

The figures are for purposes of illustrating example embodiments, but it is understood that the inventions are not limited to the arrangements and instrumentality shown in the drawings. In the figures, identical reference numbers identify at least generally similar elements.

### DETAILED DESCRIPTION

The present disclosure describes systems, methods, and apparatuses configured to provide suction for patients to assist with dysphagia and hypersalivation. Conventional devices used to provide suction for patients to assist with dysphagia and hypersalivation, even those branded as “compact” or “portable,” are large and cumbersome. Most conventional suction devices have dimensions of close to, or even more than, a foot and weigh multiple pounds. Further, they are conspicuous and difficult, if not impossible, for patients to use. Accordingly, there is a need for a compact, portable, discreet, handheld, and user-controlled suction device to assist, but not limited to people with swallowing disorders to evacuate liquid waste from the oral cavity without outside assistance.

The present disclosure will now be described more fully hereinafter with reference to the accompanying drawings, which form a part hereof, and which show, by way of non-limiting illustration, certain examples. Subject matter may, however, be described in a variety of different forms and, therefore, covered or claimed subject matter is intended to be construed as not being limited to any examples set forth herein. Among other things, subject matter may be described as methods, devices, components, or systems. Accordingly, examples may take the form of hardware, software, firmware, or any combination thereof (other than software per se). The following detailed description is, therefore, not intended to be taken in a limiting sense.

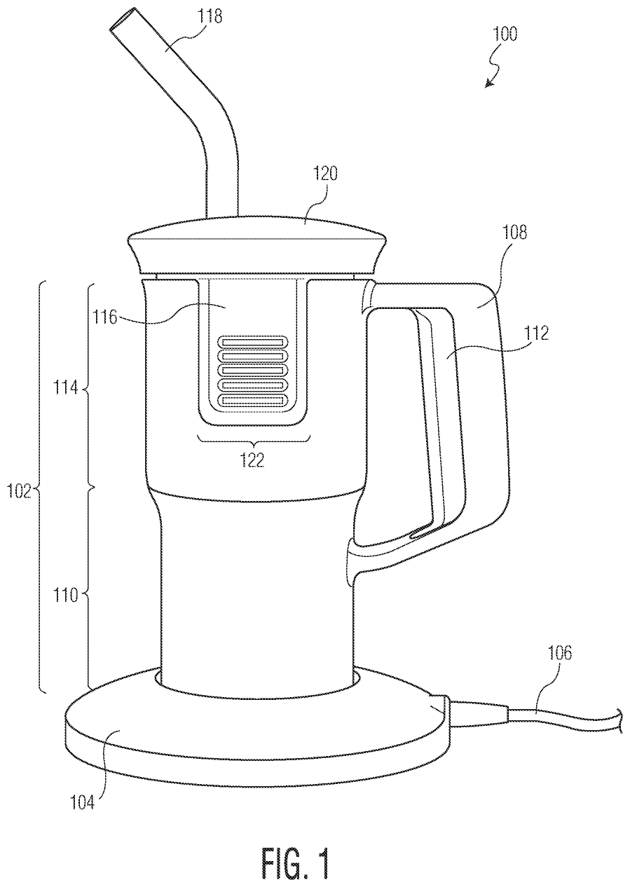

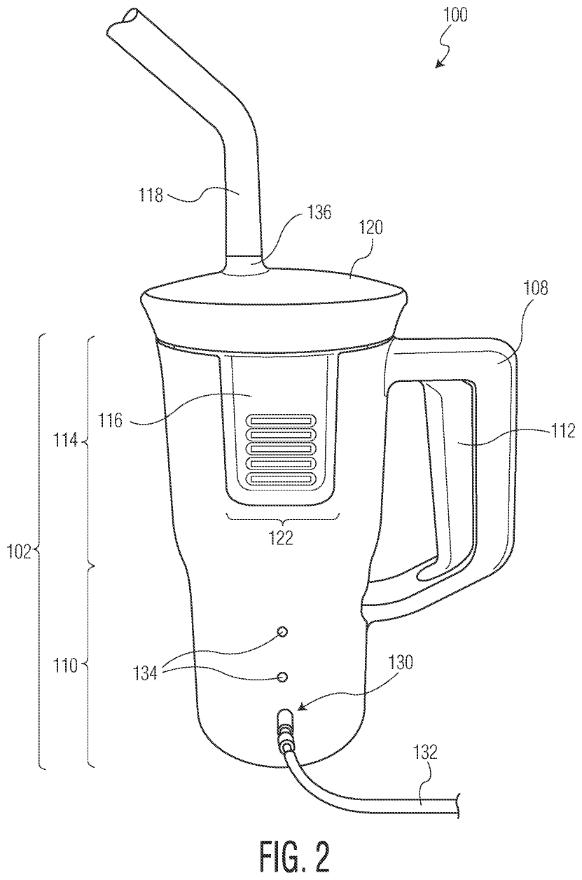

Referring now to FIGS. 1 and 2, side views of a handheld portable oral liquid vacuum aspirator device 100 is shown. The device 100 may include a main unit 102, a reservoir 116, a reservoir cap 120, and a flexible mouthpiece 118.

In an example, as shown in FIG. 1, the device 100 may be charged via a charging base 104 that is connected to a charging base power cord 106. In an example, the base power cord 106 may be permanently affixed to the charging base 104. In another example, the base power cord 106 may be removable and may plug into a proprietary or conventional (e.g., USB) port located on the charging base.

The charging base 104 may be countered to receive a lower portion 110 of the main unit 102 and serve as a table-top cup holder. In an example, the charging base 104 may include contact points that correspond to contact points on the main unit 102 to enable electricity from the power cord 106 to travel through the charging base 104 to a battery within in the main unit 102 for charging. In another example, the main unit 102 may include circuitry (e.g., a magnetic coil) configured to use inductive charging and/or wirelessly charging to charge the battery within the main unit 102. In an example, the charging base 104 and/or the main unit 102 may have one or more indicators (e.g., lights) that provide information about charging of the main unit 102.

In another example, as shown in FIG. 2, the device 100 may be charged via a port 130 located on one or more of a lower portion 110 and an upper portion 114 of the main unit 102. In this example, all battery and charging components (e.g., removable primary cell and/or secondary cell batteries) may be included within the main unit 102 and the main unit 102 may be powered by a power cord 132 that may be removably connected to the main unit 102 via the proprietary or conventional (e.g., USB) port 130. In an example, the main unit 102 may have one or more indicators 134 (e.g., lights) that provide information about charging of the main unit 102.

The main unit 102 may be a discreet mug-shaped unit with the lower portion 110 that is contoured/shaped to fit a cup holder (e.g., in a vehicle, walker, or wheelchair) and a handle 108 that facilitates one handed operation by users. As described in additional detail below, the lower portion 110 may contain on or more components that enable vacuum operation (e.g., a pump, a battery, and circuitry). In an example, the handle 108 may include a trigger 112 that is configured to provide a user-friendly on/off switch that can be operated by the user with one hand. In another example, the handle 108 may include one or more buttons to control operation of the main unit 102. In an example, the handle 108 may be connected to the lower portion 110. In another example, the handle 108 may be connected an upper portion of the main unit 102. In another example, the handle 108 may be connected to the lower portion 110 and the upper portion 114.

The upper portion 114 of the main unit 102 may house a removable, and optionally disposable, reservoir 116 to collect any liquid suctioned through the flexible mouthpiece 118. The upper portion 114 may have a larger diameter than the lower portion 110. The reservoir 116 may be configured to be easy to clean and to keep sterile. In an example, as shown in FIG. 1, the flexible mouthpiece 118 may be coupled to, and extend through, a reservoir cap 120 in an air-tight manner. In another example, as shown in FIG. 2, the flexible mouthpiece 118 may fit over a protrusion 136 from the reservoir cap 120 (with or without the use of a gasket around the protrusion 136) in an air-tight manner.

The flexible mouthpiece 118 may be removable, and optionally disposable. The flexible mouthpiece 118 may be composed, at least partially, of silicone. In an example, one or more of the reservoir 116 and the flexible mouthpiece 118 may include a filter membrane. The reservoir cap 120 may be affixed (e.g., screwed) to the reservoir 116 in an air-tight manner. The upper portion 114 may have an opening 122 through which the reservoir 116 is visible, such that a liquid level within the reservoir 116 is visible. At least a portion of the reservoir 116 that is visible through the opening 122 may be semi-transparent (e.g., frosted) such that the liquid level is visible, but still somewhat concealed from easy view. This portion of the reservoir 116 may also have markings to show the fluid level.

The components of the device 100 may be of any size and volume. In a non-limiting example, the device 100 may have a height of approximately 6 in. The charging base 104 may have a height of approximately 1 in. The upper portion 114 may have a circumference of approximately 11 in. The lower portion 110 may have a circumference of approximately 4 in. The reservoir 116 may have a high of approximately 4 in and a circumference of approximately 10.75 in to fit snugly, but not too tightly, in the main unit 102. The reservoir 116 may have a volume ranging from approximately 200 ml to approximately 500 ml. Overall, the main unit 102, the reservoir 116, the reservoir cap 120, and the flexible mouthpiece 118 may have a dry weight of approximately 12.8 oz. Any and all other sizes, dimensions, and volumes of the device 100 and its components are contemplated and within the scope of the present disclosure.

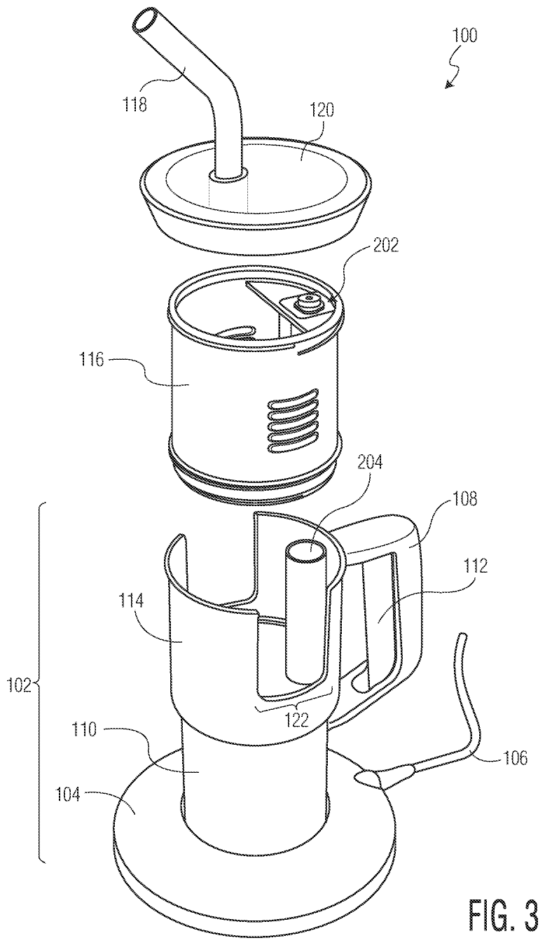

Referring now to FIG. 3, an exploded view of the device 100 is shown. As shown in FIG. 2, the reservoir cap 120 may be removable from the reservoir 116. In an example, the reservoir cap 120 and the reservoir 116 may have corresponding threads to allow the reservoir cap 120 to be screwed onto the reservoir 116. The reservoir cap 120 may be configured to “click” into place when it is fully sealed. In another example, the reservoir cap 120 may be press fit onto the reservoir 116. In any method used, the seal between the reservoir cap 120 and the reservoir 116 may be air/liquid tight. The reservoir 116 may fit into the upper portion 114 of the main unit 102. The reservoir 116 may include a valve portion 202 that fits into a vacuum passageway 204.

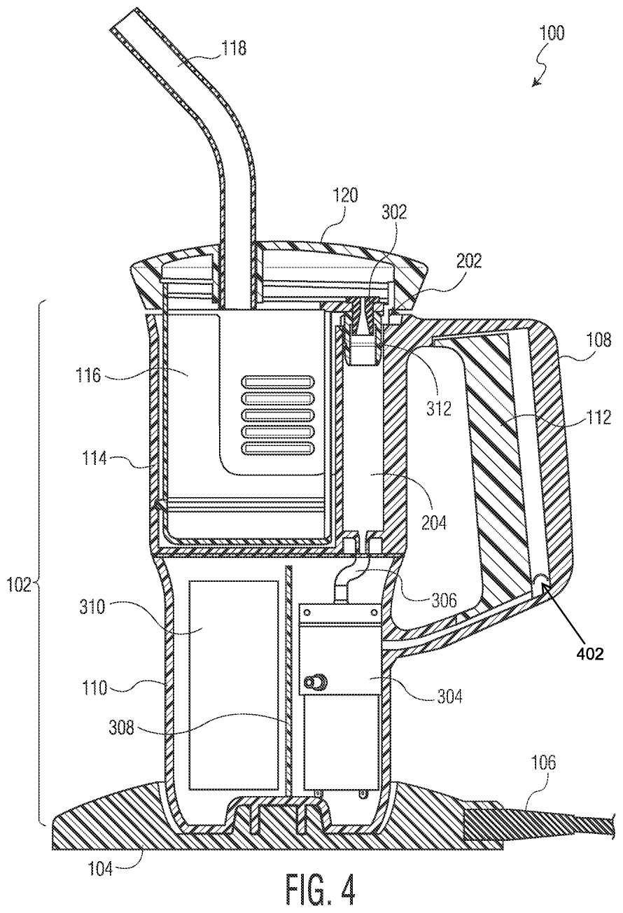

Referring now to FIG. 4, a cross section view of the device 100 is shown. As described above, the valve portion 202 of the reservoir 116 may fit into the vacuum passageway 204. The valve portion 202 may include a one-way valve 302 that allows negative pressure to flow from a vacuum pump 304, through a connecting hose 306, through the vacuum passageway 204, and into the reservoir 116 without letting any liquids flow back from the reservoir 116 into the vacuum passageway 204. The valve portion 202 may be secured in the vacuum passageway 204 by an O-ring 312 that creates a water/airtight seal. Operation of the vacuum pump 304 may be controlled by circuitry mounted on a substrate 308. The vacuum pump 304 may provide a negative pressure ranging from approximately 40 mm Hg to approximately 200 mm Hg. In an example, the substrate 308 may be a printed circuit board (PCB). The circuity on the substrate 308 may be coupled to one or more of the vacuum pump 304, the trigger 112, and a battery 310. The battery 310 may also be coupled directly to the vacuum pump 304.

In operation, a user may pick up the main unit 102 by the handle and press the trigger 112 to activate the vacuum pump 304. In an example, the device 100 may be configured so this may be done simultaneously. In an example, a resistance device 402, such as a spring, may be used to provide resistant to the trigger 112. The trigger 112 and or the resistance device 402 may be electrically coupled to one or more of the circuitry mounted on a substrate 308, the vacuum pump 304, and the battery 310. The user may place the flexible mouthpiece 118 in his/her mouth to suction out excessive liquids. Once the liquids are removed, the user may either release the trigger 112 or put the main unit 102 down, either of which will automatically stop suction from the vacuum pump 304. Optionally, the user may place the main unit 102 into the charging base 104 to recharge the battery 310. Once fluid in the reservoir 116 has reached a certain level (e.g., approximately 6-8 oz) the vacuum pump 304 will no longer be able to suction. The user and/or a caregiver may then remove the reservoir 116 to empty and/or replace it.

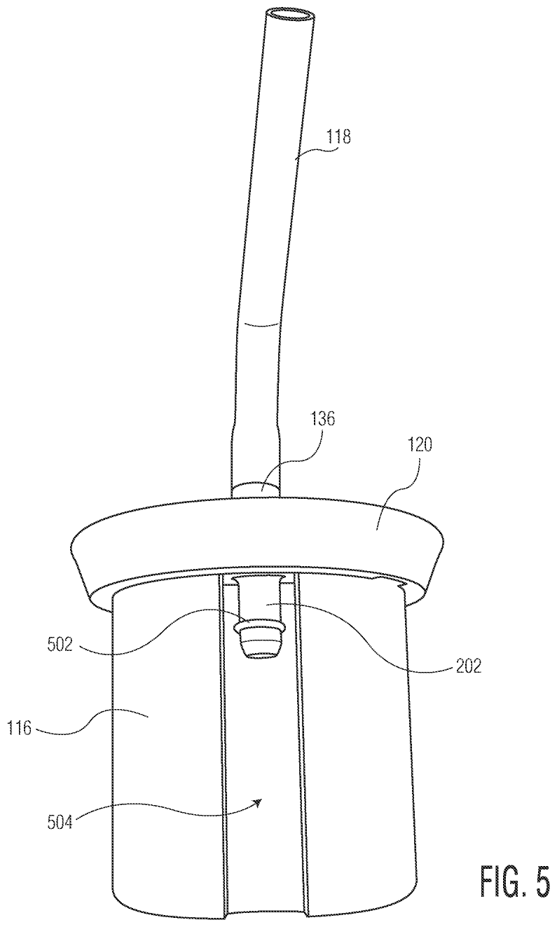

Referring now to FIG. 5, a side view of the reservoir 116 is shown. FIG. 5 shows the valve portion 202 of the reservoir 116 that may fit into the vacuum passageway 204. The valve portion 202 may extend into the vacuum passageway 204 and may include a gasket 502 to ensure an air-tight seal. The reservoir 116 may have a contour 504 to accommodate the vacuum passageway 204.

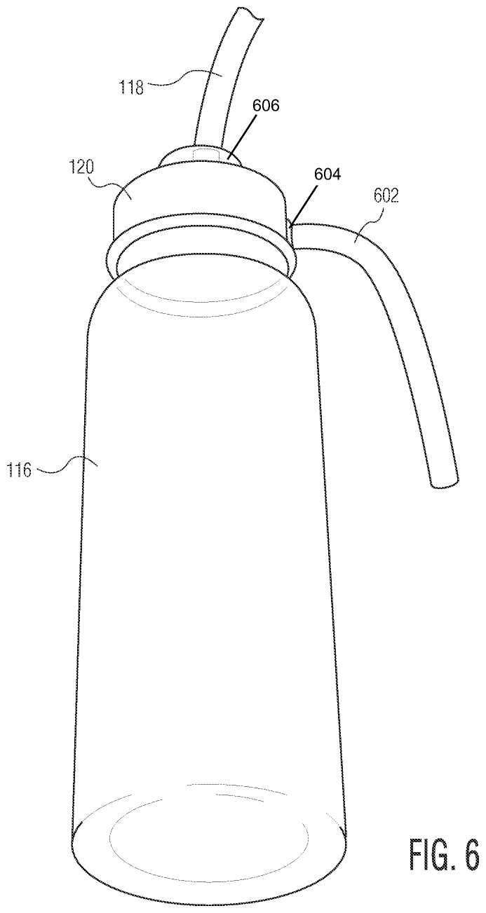

Referring now to FIG. 6, a side view of another example of the reservoir 116 is shown. In this example, rather than connecting directly to the vacuum passageway 204 through the valve portion 202, the reservoir 116 may be connected to one or more of the vacuum passageway 204 or directly to the vacuum pump 304 via a vacuum hose 602. The vacuum hose 602 may be coupled to, and extend through, the reservoir cap 120 in an air-tight manner. In an example, a first seal 604 may ensure the vacuum hose 602 is coupled to the reservoir cap 120 in an air-tight manner. The flexible mouthpiece 118 may extend through the reservoir cap 120 in an air-tight manner. In an example, a second seal 606 may ensure the flexible mouthpiece 118 is coupled to the reservoir cap 120 in an air-tight manner.

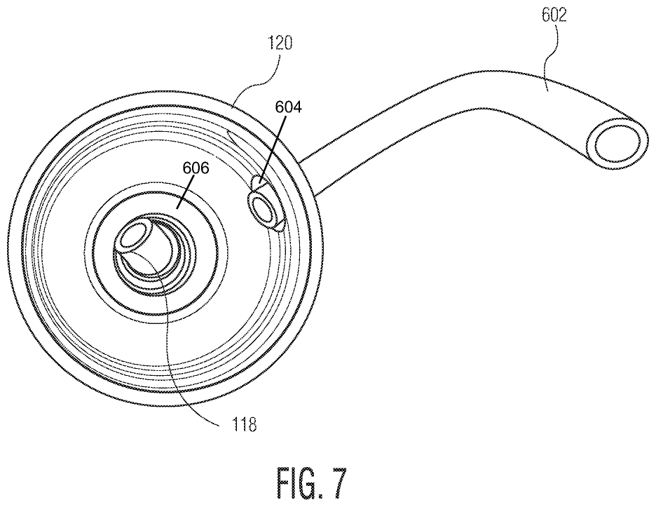

Referring now to FIG. 7, a bottom view of the reservoir cap 120 of FIG. 6 is shown. FIG. 7 shows the coupling of the flexible mouthpiece 118 and the vacuum hose 602 to the reservoir cap 120.

Referring now to FIG. 8, a perspective view of the reservoir 116 of FIG. 6 within the upper portion 114 of the device 100 is shown. In this example, the device 100 may include a separate device lid 802 that fits over the upper portion 114 and conceals the reservoir 116 and the reservoir cap 120. The flexible mouthpiece 118 may extend through the device lid 802. The upper portion 114 may or may not include the opening 122 through which the reservoir 116 is visible as shown in FIG. 1.

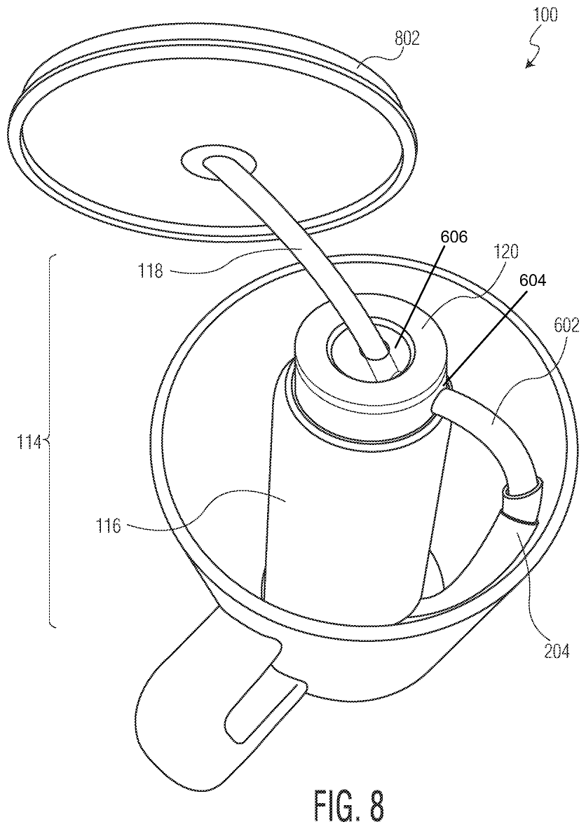

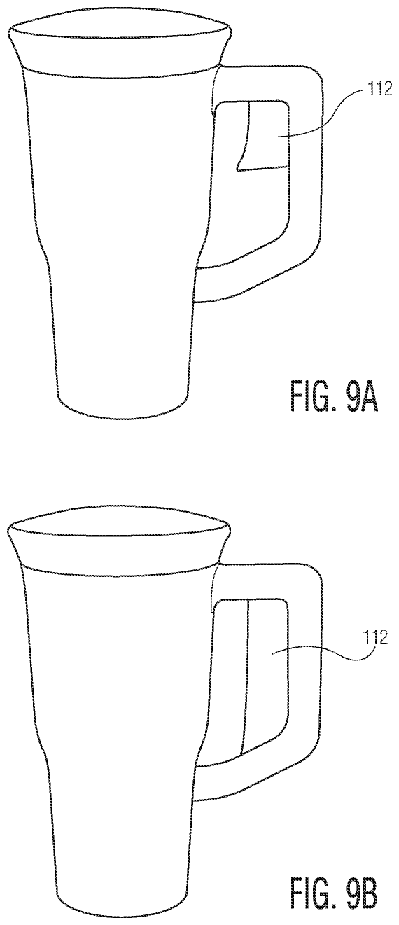

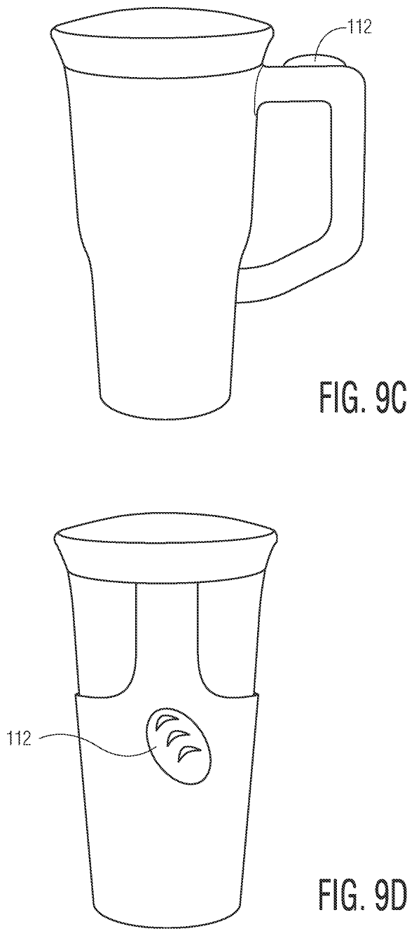

Referring now to FIGS. 9A-9D, cross section views of different examples of triggers 112 that may be used are shown. FIG. 9A shows an example in which the trigger 112 covers only a portion of an inner surface of the handle 108. In this example, the trigger 112 may be configured to be operated with a single finger. FIG. 9B shows an example in which the trigger 112 covers the entire portion of the inner surface of the handle 108. In this example, the trigger 112 may be configured to be operated with one or more fingers (e.g., all four fingers). FIG. 9C shows an example in which the trigger 112 is covers only a portion of an outer surface of the handle 108. In this example, the trigger 112 may be configured to be operated with a user's thumb. FIG. 9D shows an example of a main unit 102 without a handle 108. In this example, the trigger 112 may be operated when gripping the main unit 102.

Additional examples of the presently described method and device embodiments are suggested according to the structures and techniques described herein. Other non-limiting examples may be configured to operate separately or may be combined in any permutation or combination with any one or more of the other examples provided above or throughout the present disclosure.

It will be appreciated by those skilled in the art that the present disclosure may be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The terms “including” and “comprising” should be interpreted as meaning “including, but not limited to.” If not already set forth explicitly in the claims, the term “a” should be interpreted as “at least one” and the terms “the, said, etc.” should be interpreted as “the at least one, said at least one, etc.”

It is understood that the circuitry mounted on a substrate 308 may include analog or digital hardware and computer program instructions stored on a non-transitory computer readable medium. These computer program instructions may be provided to a processor of a general-purpose computer to alter its function as detailed herein, a special purpose computer, ASIC, or other programmable data processing apparatus, such that the instructions, which execute via the processor of the computer or other programmable data processing apparatus, implement the functions/acts specified.

For the purposes of this disclosure, non-transitory computer readable medium (or computer-readable storage medium/media) stores computer data, which data may include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable, and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, cloud storage, magnetic cassettes, magnetic tape, magnetic disk storage or other magnetic storage devices, or any other physical or material medium which may be used to tangibly store the desired information or data or instructions and which may be accessed by a computer or processor.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

It is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A handheld portable oral aspirator device comprising:
a lower portion of a main unit, the lower portion comprising a battery, circuitry mounted on a substrate, and a vacuum pump;
an upper portion of the main unit, the upper portion configured to house a reservoir coupled to one of a vacuum pump and a vacuum passageway connected to the vacuum pump via a hose;
a reservoir cap configured to be affixed to one of the reservoir and the upper portion;
a flexible mouthpiece coupled to the reservoir cap; and
a handle coupled to one or more of the lower portion and the upper portion, the handle comprising a trigger that is configured to cause the circuitry to activate the vacuum pump only when pressed.

2. The device of claim 1, further comprising:
a charging base configured to receive the lower portion, the charging base comprising charging circuitry configured to charge the battery.

3. The device of claim 2, wherein the charging circuitry is configured to charge the battery via one or more of wireless or inductive charging.

4. The device of claim 1, further comprising a charging port on one of the lower portion and the upper portion coupled to the battery.

5. The device of claim 1, wherein the upper portion has a larger diameter than the lower portion.

6. The device of claim 1, wherein the reservoir is removable.

7. The device of claim 1, wherein the upper portion has an opening through which the reservoir is visible.

8. The device of claim 7, wherein at least a portion of the reservoir visible through the opening is semi-transparent.

9. The device of claim 1, wherein the reservoir is coupled to the vacuum passageway via a valve portion of the reservoir that extends into the vacuum passageway.

10. The device of claim 9, wherein the valve portion comprises a one-way valve configured to allow negative pressure to flow from the vacuum pump, through the hose, through the vacuum passageway, and into the reservoir without letting any liquids flow back from the reservoir.

11. The device of claim 9, wherein the valve portion comprises an o-ring configured to create a water/airtight seal between the valve portion and the vacuum passageway.

12. The device of claim 1, wherein the reservoir is coupled to one of the vacuum pump and the vacuum passageway via a vacuum hose that extends through the reservoir cap.

13. The device of claim 12, further comprising:
a device lid configured to fit over the upper portion and conceal the reservoir and the reservoir cap, wherein the flexible mouthpiece extends through the device lid.

14. The device of claim 1, wherein the reservoir cap is configured to one of press fit onto the reservoir and screw onto the reservoir via threads.

15. The device of claim 11, wherein the reservoir cap is configured to “click” when fully tightened.

16. The device of claim 1, wherein the trigger covers one of an entire portion of an inner surface of the handle, only a portion of an inner surface of the handle, and only a portion of an outer surface of the handle.

17. The device of claim 1, wherein the flexible mouthpiece extends through the reservoir cap in an air-tight manner.

18. The device of claim 1, wherein the flexible mouthpiece fits over a protrusion from the reservoir cap in an air-tight manner.

19. The device of claim 1, wherein the vacuum pump is configured to create a negative pressure ranging from approximately 40 mm Hg to approximately 200 mm Hg.

20. The device of claim 1, wherein the reservoir has a volume ranging from approximately 200 ml to approximately 400 ml.
